(19) United States
(12) Patent Application Publication
(10) Pub. No.: US 2017/0113069 A1
(43) Pub. Date: Apr. 27, 2017

(54) ULTRASONIC ENERGY THERAPY DEVICE AND ULTRASONIC ENERGY THERAPY METHOD

(71) Applicant: OLYMPUS CORPORATION, Tokyo (JP)

(72) Inventor: Sadao EBATA, Tokyo (JP)

(73) Assignee: OLYMPUS CORPORATION, Tokyo (JP)

(21) Appl. No.: 15/401,464

(22) Filed: Jan. 9, 2017

Related U.S. Application Data
(63) Continuation of application No. PCT/JP2015/057255, filed on Mar. 12, 2015.

(30) Foreign Application Priority Data
Jul. 18, 2014 (JP) 2014-147802

(51) Int. Cl. A61N 7/00; A61B 8/06; A61B 8/02

(52) U.S. Cl. CPC A61N 7/00; A61B 8/02; A61B 8/06; A61N 2007/0047

(57) ABSTRACT

An ultrasonic energy therapy device including: an insertion portion that has an elongated shape and is insertable into a blood vessel; a piezoelectric element that is attached to the insertion portion and radiates ultrasonic energy from inside of the blood vessel toward a biological tissue outside the blood vessel; a thermometric sensor; a temperature detection section that detects the temperature of the thermometric sensor; and a control section that controls the piezoelectric element depending on the temperature of the thermometric sensor measured by the temperature detection section so that the ultrasonic energy in a desired amount is radiated to the biological tissue.

### CROSS-REFERENCE TO RELATED APPLICATIONS

This is a continuation of International Application PCT/JP2015/057255, with an international filing date of Mar. 12, 2015, which is hereby incorporated by reference herein in its entirety.

This application is based on Japanese Patent Application No. 2014-147802, the contents of which are incorporated herein by reference.

### TECHNICAL FIELD

The present invention relates to an ultrasonic energy therapy device and an ultrasonic energy therapy method.

### BACKGROUND ART

An ultrasonic energy therapy device is known to treat a lesion part by radiating ultrasonic energy to a biological tissue in the related art (for example, see PTL 1). The ultrasonic energy therapy device disclosed in PTL 1 radiates the ultrasonic energy from an ultrasonic radiation surface of an insertion portion inserted into a blood vessel toward a blood vessel wall while maintaining a distance between the ultrasonic radiation surface and the blood vessel wall at a desired distance by a wire, a spring or the like to radiate the ultrasonic energy to the lesion part with accuracy.

### CITATION LIST

### Patent Literature

{PTL 1} PCT International Publication No. WO 2012/052924

### SUMMARY OF INVENTION

A first aspect of the present invention is directed to an ultrasonic energy therapy device comprising: an insertion portion that has an elongated shape and is insertable into a blood vessel; an energy emitter that is attached to the insertion portion and emits ultrasonic energy from inside of the blood vessel toward a biological tissue outside the blood vessel; a loss amount measurement device that measures a loss amount of ultrasonic energy emitted from the energy emission section caused by a blood flow; and a controller that controls the energy emission section based on the loss amount measured by the loss amount measurement section so that the ultrasonic energy in a desired amount is radiated to the biological tissue.

A second aspect of the present invention is directed to an ultrasonic energy therapy method comprising: an energy emission step of emitting ultrasonic energy from inside of a blood vessel toward a biological tissue outside the blood vessel; and a loss amount measurement step of measuring a loss amount of ultrasonic energy emitted in the energy emission step caused by blood flow, wherein the energy emission step adjusts an emission of the ultrasonic energy according to the loss amount measured in the loss amount measurement step so that the ultrasonic energy in a desired amount is radiated to the biological tissue.

A third aspect of the present invention is directed to an ultrasonic energy therapy method comprising: an energy emission step of emitting ultrasonic energy from inside of a blood vessel toward a biological tissue outside the blood vessel; and a loss value detection step of detecting a change in a loss value with time of the ultrasonic energy emitted in the energy emission step caused by blood flow, wherein when the loss value detected in the loss value detection step is reduced, the energy emission step lowers an intensity of the ultrasonic energy and/or shortens an emission time of the ultrasonic energy, and wherein when the detected loss value is increased, the energy emission step increases the intensity of the ultrasonic energy and/or prolongs the emission time of the ultrasonic energy.

### BRIEF DESCRIPTION OF DRAWINGS

FIG. 1 is a block diagram illustrating an ultrasonic energy therapy device according to a first embodiment of the present invention.

FIG. 2 is a diagram illustrating an insertion portion of the ultrasonic energy therapy device in FIG. 1 which is inserted into a blood vessel when being viewed in a radial direction thereof and in a longitudinal direction thereof.

FIG. 3 is a flow chart illustrating an ultrasonic energy therapy method according to the first embodiment of the present invention.

FIG. 4 is timing charts indicating the relations between time and a change in blood flow in the vicinity of a thermometric sensor, a detection temperature of the thermometric sensor, a waveform of a detection temperature input to a smoothing circuit section, and a waveform of the detection temperature output from the smoothing circuit section, respectively.

FIG. 5 is a flow chart illustrating an ultrasonic energy therapy method according to one modification of the first embodiment of the present invention.

FIG. 6 is a block diagram illustrating an ultrasonic energy therapy steps according to a second embodiment of the present invention.

FIG. 7 is a diagram illustrating a pulsation cycle detection section of an ultrasonic energy therapy device according to the second embodiment of the present invention.

FIG. 8 is a flow chart illustrating ultrasonic energy therapy steps according to the second embodiment of the present invention.

FIG. 9 is timing charts indicating the relations between time and a change in blood flow in the vicinity of a thermometric sensor, a detection temperature of the thermometric sensor, an output signal of a comparator, a pulsation cycle pulse, and an output of the ultrasonic energy, respectively.

FIG. 10 is a diagram illustrating an insertion portion of an ultrasonic energy therapy device according to a third embodiment of the present invention which is inserted into a blood vessel when being viewed in a radial direction thereof and in a longitudinal direction thereof.

FIG. 11 is a block diagram illustrating the ultrasonic energy therapy device in FIG. 10.

FIG. 12 is a diagram illustrating an upstream thermometric sensor determination section and a time measurement section in FIG. 11.

FIG. 13 is timing charts indicating changes in detection temperatures with time of two thermometric sensors.

FIG. 14 is timing charts indicating the relations between time and a detection temperature of a thermometric sensor 13A, a detection temperature of a thermometric sensor 13B, an output of a pulsation cycle detection section 41A, an output of a pulsation cycle detection section 41B, a differential time signal between the pulsation cycle detection sections 41A and 41B, a pulsation cycle pulse, and an output of the ultrasonic energy, respectively, where the thermometric sensor 13A is disposed upstream in a blood flow direction.

FIG. 15 is timing charts indicating the relations between time and a detection temperature of a thermometric sensor 13B, a detection temperature of a thermometric sensor 13A, an output of the thermometric sensor 13B, an output of the thermometric sensor 13A, a differential time signal between the pulsation cycle detection sections 41A and 41B, a pulsation cycle pulse, and an output of the ultrasonic energy, respectively, where the thermometric sensor 13B is disposed upstream in the blood flow direction.

FIG. 16 is a flow chart illustrating ultrasonic energy therapy steps according to a third embodiment of the present invention.

FIG. 17 is a diagram illustrating an insertion portion of an ultrasonic energy therapy device according to a modification of each embodiment of the present invention which is inserted into a blood vessel when being viewed in a radial direction thereof and in a longitudinal direction thereof.

### DESCRIPTION OF EMBODIMENTS

### First Embodiment

An ultrasonic energy therapy device and an ultrasonic energy therapy method according to a first embodiment of the present invention will be described below with reference to the accompanying drawings.

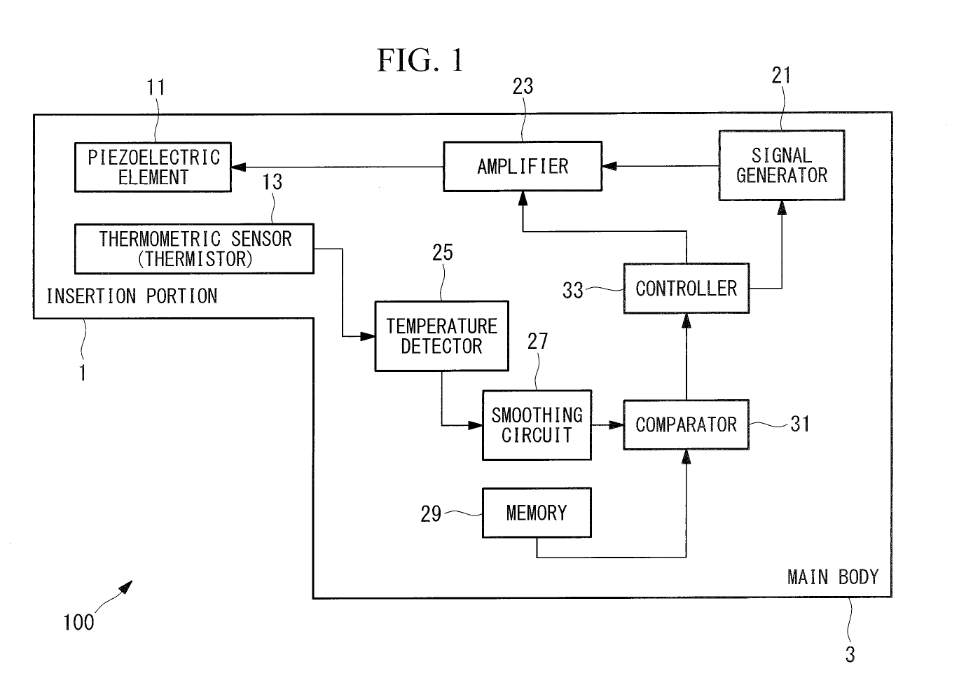

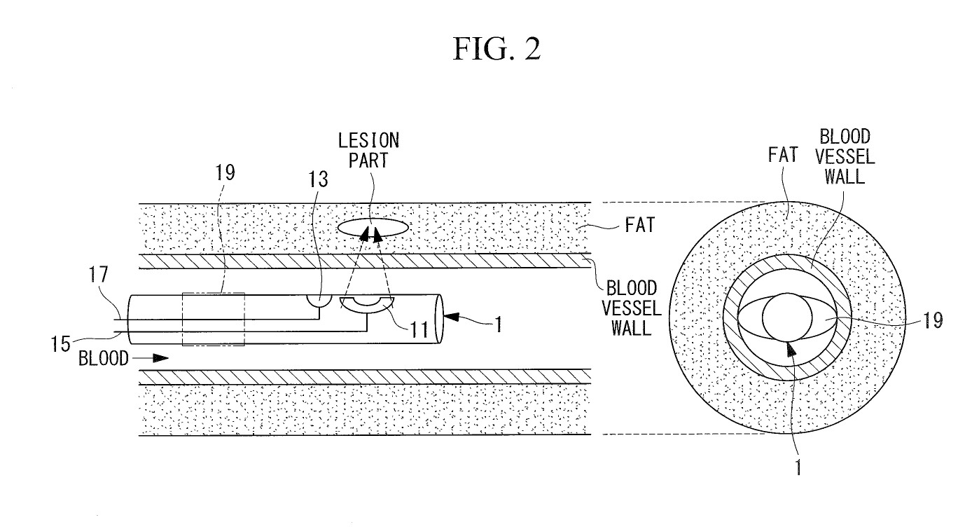

An ultrasonic energy therapy device 100 according to the present embodiment comprises: an insertion portion 1 that has an elongated and substantially cylindrical shape and is insertable into a blood vessel of a patient; and a main body 3 that supports the insertion portion 1 as illustrated in FIG. 1 and FIG. 2.

The insertion portion 1 comprises: a piezoelectric element (energy emitter) 11 that is configured to generate ultrasonic energy; and a thermometric sensor (energy loss measurement section) 13 such as a thermistor that can detect the velocity of blood flow in the blood vessel.

The piezoelectric element 11 can generate the ultrasonic energy from an emission surface formed in a concave face so that the ultrasonic energy is focused at high density. The ultrasonic energy emitted from the piezoelectric element 11 can be changed to thermal energy at a focal position focused on a lesion part in a biological tissue, thereby the lesion part can be treated by being heated or cauterized. The piezoelectric element 11 is attached to the insertion portion 1 with the emission surface facing outward in a radial direction of the insertion portion 1, and is connected to the main body 3 through a signal line 15.

The thermometric sensor 13 is connected to the main body 3 through the signal line 17 and can generate heat by energization. In the thermometric sensor 13, the generated heat is taken away by cooling effect of the blood flow, thereby increasing an electric resistance value.

Balloons 19 are attached to the insertion portion 1 so that the insertion portion 1 is fixed in a positioning state in the blood vessel. The balloons 19 are disposed on a proximal end side of the insertion portion 1 than the piezoelectric element 11 and the thermometric sensor 13. When these balloons 19 are filled with gas or liquid, they can be expanded radially outward from two places of the insertion portion 1, respectively, the two places being shifted by 180° from each other in a circumferential direction of the insertion portion 1. Thus, the balloons 19 can be expanded in two directions opposite to each other from the insertion portion 1 in the blood vessel to be contacted with a blood vessel wall, thereby the insertion portion 1 can be fixed in the positioning state in the radial direction without interrupting the blood flow.

The main body 3 comprises: a signal generator 21 that can be generate a reference waveform signal of power; an amplifier 23 that can amplify the reference waveform signal generated by the signal generation section 21 to apply to the piezoelectric element 11; a temperature detector (loss amount measurement device) 25 that can detect a temperature of the thermometric sensor 13; a smoothing circuit 27 that can smooth a waveform of the detection temperature detected by the temperature detection section 25; a memory 29 that can store a predetermined threshold relating to the temperature; a comparator 31 that can compare the detection temperature smoothed by the smoothing circuit section 27 and the predetermined threshold stored in the storage section 29; and a controller 33 that can control the signal generation section 21 and the amplification section 23 based on a comparison result by the comparison section 31.

The temperature detection section 25 can measure weak electric current supplied to the thermometric sensor 13 to measure the electric resistance value of the thermometric sensor 13. In the thermometric sensor 13, heat is taken away, and the electric resistance value is increased. Therefore, the temperature detection section 25 can detect the temperature of the thermometric sensor 13 indirectly, by measuring the resistance value of the thermometric sensor 13. Since a rising rate of the electric resistance value of the thermometric sensor 13 has a unique relationship with a flow velocity of fluid, the temperature detection section 25 can detect the velocity of the blood flow by measuring the electric resistance value of the thermometric sensor 13. A loss amount of ultrasonic energy caused by the blood flow can be measured based on the velocity of the blood flow.

Thus, the thermometric sensor 13 and the temperature detection section 25 can measure the loss amount of ultrasonic energy caused by the blood flow indirectly, by detecting the temperature of the thermometric sensor 13. The temperature detection section 25 can send a detection result of the measured electric resistance value of the thermometric sensor 13 as the detection temperature to the smoothing circuit section 27.

The smoothing circuit section 27 can smooth a waveform of the detection temperature sent from the temperature detection section 25 to send to the comparison section 31.

The storage section 29 can store a threshold α and a threshold β larger than the threshold α. When the detection temperature of the thermometric sensor 13 is high, that is, the loss amount of ultrasonic energy is small, it means that the blood flow is slow, and thermal energy taken off by the blood flow out of the ultrasonic energy emitted from the piezoelectric element 11 is small. In this case, the amount of ultrasonic energy radiated to the biological tissue is not insufficient. On the other hand, when the detection temperature of the thermometric sensor 13 is low, that is, the loss amount of ultrasonic energy is large, it means that the blood flow is fast, and thermal energy taken off by the blood flow out of the ultrasonic energy emitted from the piezoelectric element 11 is large. In this case, the amount of ultrasonic energy radiated to the biological tissue is insufficient. Therefore, the storage section 29 stores, as the threshold α, the lowest value of the detection temperature of the thermometric sensor 13 in a situation where the amount of ultrasonic energy radiated to the biological tissue is sufficient.

When the detection temperature of the thermometric sensor 13 is very high, that is, the loss amount of ultrasonic energy is very small, the blood flow does not affect substantially and a distance between the insertion portion 1 and the blood vessel wall may not be maintained at a desired distance interval. In this case, the amount of ultrasonic energy radiated to the biological tissue becomes excessive. Therefore, the storage section 29 stores, as the threshold β, the highest value of the detection temperature of the thermometric sensor 13 in a situation where the distance between the insertion portion 1 and the blood vessel wall is maintained at the desired distance interval.

When the detection temperature of the thermometric sensor 13 is replaced with the loss amount of ultrasonic energy, the maximum loss amount of ultrasonic energy corresponding to the threshold α in the situation where the amount of ultrasonic energy radiated to the biological tissue is sufficient is determined as a threshold γ (a first threshold), and the minimum loss amount of ultrasonic energy corresponding to the threshold β in the situation where the distance between the insertion portion 1 and the blood vessel wall is maintained at the desired distance interval is determined as a threshold δ (a second threshold), a relation of the threshold γ>the threshold δ is satisfied. Therefore, a high and low relation between the threshold α and the threshold β is the reverse of a magnitude relation between the threshold γ and the threshold δ.

The comparison section 31 compares the detection temperature of the thermometric sensor 13 that is smoothed by and sent from the smoothing circuit section 27 and the threshold α stored in the storage section 29, and sends a comparison result to the control section 33. When the comparison section 31 determines that the detection temperature of the thermometric sensor 13 is equal to or more than the threshold α, the comparison section 31 compares the detection temperature of the thermometric sensor 13 and the threshold β and sends the comparison result to the control section 33.

When the comparison section 31 determines that the detection temperature of the thermometric sensor 13 is lower than the threshold α, that is, the loss amount of ultrasonic energy by the blood flow is larger than the above described threshold γ, the control section 33 controls the signal generation section 21 to prolong the emission time of the ultrasonic energy so that the ultrasonic energy in a desired amount is radiated to the biological tissue.

When the comparison section 31 determines that the detection temperature of the thermometric sensor 13 is equal to or more than the threshold α, that is, the loss amount of ultrasonic energy by the blood flow is equal to or less than the threshold γ, the control section 33 controls to shorten the emission time of the ultrasonic energy from the signal generation section 21 so that the ultrasonic energy in a desired amount is radiated to the biological tissue.

When the comparison section 31 determines that the detection temperature of the thermometric sensor 13 is equal to or more than the threshold β, that is, the loss amount of ultrasonic energy by the blood flow is equal to or less than the threshold δ, the control section 33 controls the signal generation section 21 to stop the radiation of the ultrasonic energy.

Next, an ultrasonic energy therapy method according to the present embodiment will be described.

The ultrasonic energy therapy method according to the present embodiment comprises: an energy emission step of emitting ultrasonic energy from inside of a blood vessel toward a biological tissue outside the blood vessel (step SA4); a temperature detection step of detecting a loss amount of ultrasonic energy emitted in the energy emission step caused by blood flow (a loss amount measurement step), that is, a temperature of the thermometric sensor 13 (step SA1); and a comparison step of comparing the detection temperature of the thermometric sensor 13 detected in the temperature detection step and a predetermined threshold (step SA2, step SA5).

The comparison step compares the detection temperature of the thermometric sensor 13 detected in the temperature detection step and the threshold α. When the comparison step determines that the detection temperature of the thermometric sensor 13 is equal to or more than the threshold α, the comparison step compares the threshold β larger than the threshold α and the detection temperature of the thermometric sensor 13.

The energy emission step adjusts the emission of the ultrasonic energy according to the detection temperature of the thermometric sensor 13 measured in the temperature detection step so that the ultrasonic energy in a desired amount is radiated to the biological tissue. Particularly, when the comparison step determines that the detection temperature of the thermometric sensor 13 is lower than the threshold α, the energy emission step prolongs the emission time of the ultrasonic energy. When the comparison step determines that the detection temperature of the thermometric sensor 13 is equal to or more than the threshold α, the energy emission step shortens the emission time of the ultrasonic energy. When the comparison step determines that the detection temperature of the thermometric sensor 13 is equal to or more than the threshold β, the energy emission step stops the radiation of the ultrasonic energy.

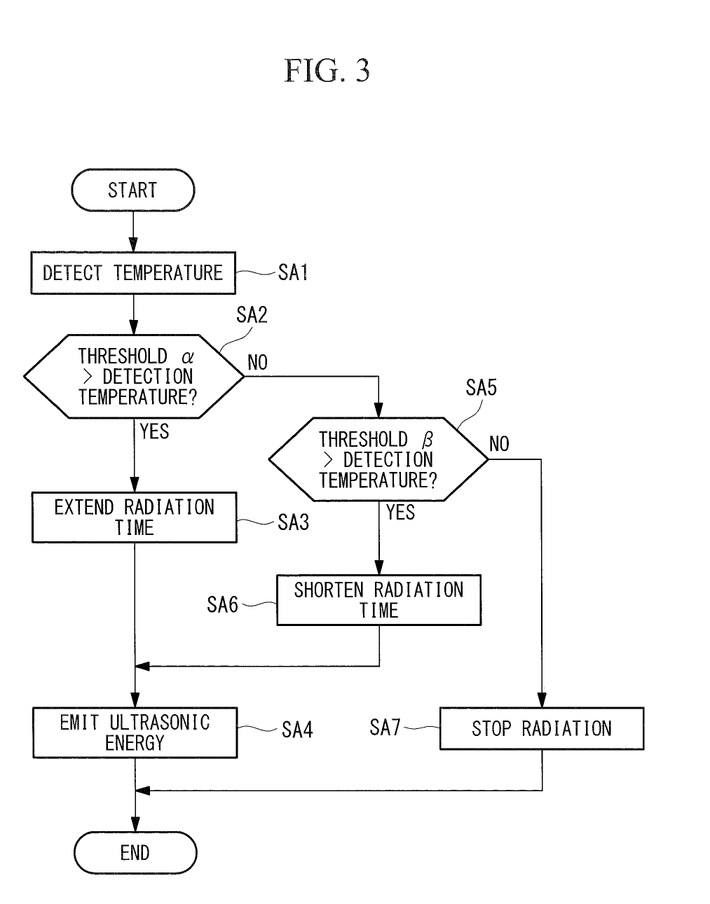

An operation of the ultrasonic energy therapy device 100 and the ultrasonic energy therapy method constituted in such a manner will be described with reference to a flow chart in FIG. 3.

To treat a lesion part of a patient by using the ultrasonic energy therapy device 100 and the ultrasonic energy therapy method according to the present embodiment, the thermometric sensor 13 is energized and the insertion portion 1 is inserted into the blood vessel of the patient.

The insertion portion 1 is disposed so that the emission surface of the piezoelectric element 11 faces a lesion part in the biological tissue through the blood vessel wall, and is fixed to this position in a positioning state by expanding the balloons 19.

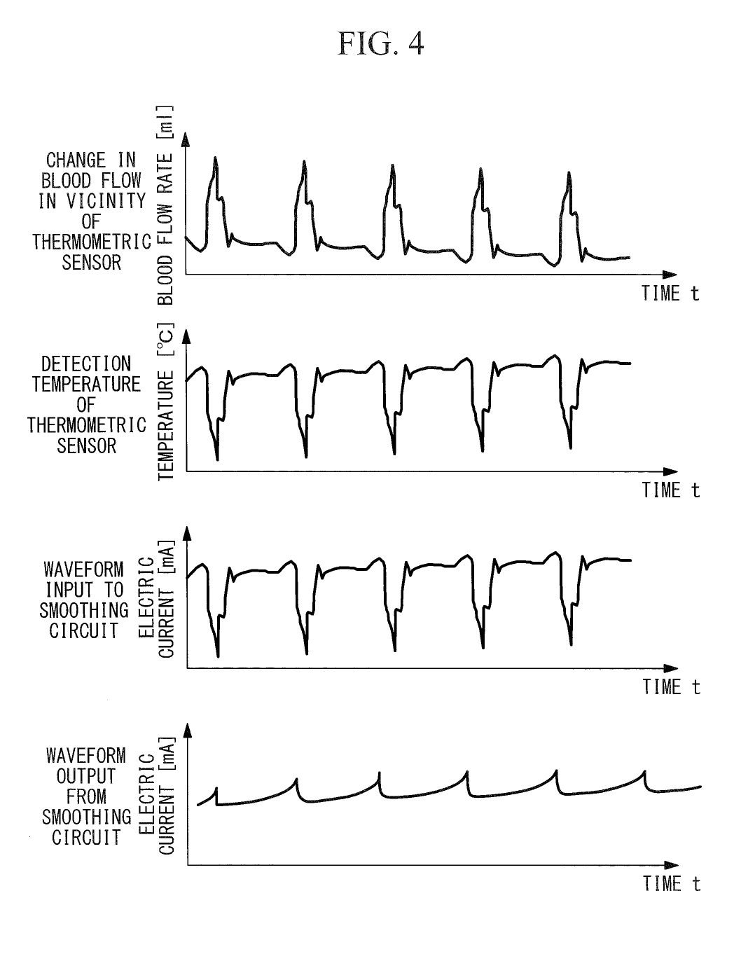

The temperature detection section 25 measures weak electric current supplied to the thermometric sensor 13 and detects the temperature of the thermometric sensor 13 (step SA1, temperature detection step). The waveform of the detection temperature of the thermometric sensor 13 detected by the temperature detection section 25 as illustrated in FIG. 4 is smoothed by the smoothing circuit section 27, and then is sent to the comparison section 31. FIG. 4 illustrates the waveforms of a change in blood flow in the vicinity of a thermometric sensor 13, a detection temperature of the thermometric sensor 13, a detection temperature input to the smoothing circuit section 27, and the detection temperature output from the smoothing circuit section 27.

The comparison section 31 compares the detection temperature of the thermometric sensor 13 sent from the smoothing circuit section 27 and the threshold α stored in the storage section 29 (step SA2, comparison step). When the comparison section 31 determines that the detection temperature of the thermometric sensor 13 is smaller than the threshold α (“Yes” in step SA2), it means that the blood flow is fast, and thermal energy taken off by the blood flow is large.

In this case, the control section 33 controls the signal generation section 21 to prolong the emission time of the ultrasonic energy emitted from the piezoelectric element 11 so that the ultrasonic energy in a desired amount is radiated to the biological tissue (step SA3). The ultrasonic energy emitted from the piezoelectric element 11 is emitted for a time longer than an initially set time (step SA4, energy emission step), thereby to compensate for the loss of ultrasonic energy caused by the blood flow and radiate the ultrasonic energy in a desired amount to the biological tissue. Thus, the lesion part can be treated sufficiently.

On the other hand, when the comparison section 31 determines that the detection temperature of the thermometric sensor 13 is equal to or more than the threshold α (“No” in step SA2), it means that the blood flow is slow, and thermal energy taken off by the blood flow is small. In this case, the comparison section 31 compares the detection temperature of the thermometric sensor 13 and the threshold β stored in the storage section 29 (step SA5, comparison step).

When the comparison section 31 determines that the detection temperature of the thermometric sensor 13 is lower than the threshold β (“Yes” in step SA5), it means that a distance interval between the insertion portion 1 and the blood vessel wall is normally maintained. In this case, the control section 33 controls the signal generation section 21 to shorten the emission time of the ultrasonic energy emitted from the piezoelectric element 11 so that the ultrasonic energy in a desired amount is radiated to the biological tissue (step SA6). The ultrasonic energy emitted from the piezoelectric element 11 is emitted for a time shorter than an initially set time (step SA4, energy emission step), the ultrasonic energy in a desired amount is radiated to the biological tissue without excessive radiation. Thus, the lesion part can be treated sufficiently.

On the other hand, when the comparison section 31 determines that the detection temperature of the thermometric sensor 13 is equal to or more than the threshold β (“No” in step SA5), it means that a distance interval between the insertion portion 1 and the blood vessel wall is not normally maintained and the insertion portion 1 is in proximity to or in contact with the blood vessel wall. In this case, the control section 33 controls the signal generation section 21 to stop the radiation of the ultrasonic energy (step SA7, energy emission step). Thus, the distance interval between the insertion portion 1 and the blood vessel wall is shifted, thereby preventing the biological tissue that is not target for treatment from being damaged by the radiation of the ultrasonic energy.

As described above, in the ultrasonic energy therapy device 100 and the ultrasonic energy therapy method according to the present embodiment, according to the waveform of the weak electric current, the control section 33 controls the emission time of the ultrasonic energy emitted from the piezoelectric element 11 so that the ultrasonic energy in a desired amount is radiated to the biological tissue, based on the detection temperature of the thermometric sensor 13, thereby, the lesion part can be sufficiently treated regardless of a difference or a change in an amount of thermal energy taken off by the blood flow. Therefore, a certain therapeutic effect can be obtained even in a case where the amount of thermal energy taken off by the blood flow differs or changes according to an individual difference, a difference in cured portion, and a difference in beat timing.

In the present embodiment, the predetermined threshold is set and the ultrasonic energy is binarized by the predetermined threshold to be radiated. Alternatively, the intensity and/or the radiation time of the ultrasonic energy may be changed seamlessly based on the flow velocity detection data, for example.

The present embodiment may be modified as follows.

That is, in the present embodiment, the control section 33 controls the emission time of the ultrasonic energy from the piezoelectric element 11 and the energy emission step adjusts the emission time of the ultrasonic energy. As one modification, the control section 33 may control the amplification section 23 to control the intensity of the ultrasonic energy emitted from the piezoelectric element 11 so that the ultrasonic energy in a desired amount is radiated to the biological tissue. The energy emission step may also adjust the intensity of the ultrasonic energy so that the ultrasonic energy in a desired amount is radiated to the biological tissue.

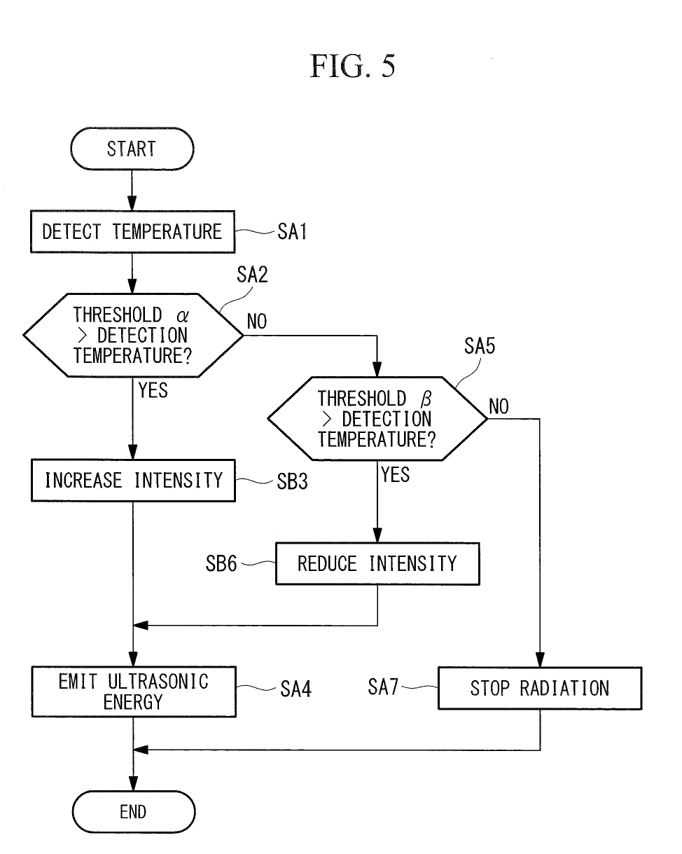

In this case, as illustrated in the flow chart in FIG. 5, in step SA2, when the comparison section 31 determines that the detection temperature of the thermometric sensor 13 is lower than the threshold α (“Yes” in step SA2), the control section 33 controls the amplification section 23, and the intensity of the ultrasonic energy emitted from the piezoelectric element 11 rises to ε (W/cm²) so that the ultrasonic energy in a desired amount is radiated to the biological tissue (step SB3). Then, the ultrasonic energy is emitted from the piezoelectric element 11 at an intensity stronger than an initially set intensity (step SA4, energy emission step), thereby to compensate for the loss of ultrasonic energy caused by the blood flow and radiate the ultrasonic energy in a desired amount to the biological tissue.

In step SA5, when the comparison section 31 determines that the detection temperature of the thermometric sensor 13 is smaller than the threshold β (“Yes” in step SA5), the control section 33 controls the amplification section 23, and the intensity of the ultrasonic energy emitted from the piezoelectric element 11 is lowered to ζ (W/cm²) so that the ultrasonic energy in a desired amount is radiated to the biological tissue (step SB6). As the intensity of the ultrasonic energy, a relation of ε>ζ is satisfied. Then, the ultrasonic energy is emitted from the piezoelectric element 11 at an intensity weaker than the initially set intensity (step SA4, energy emission step), thereby to radiate the ultrasonic energy in a desired amount to the biological tissue without excessive radiation.

In the present modification, a certain therapeutic effect can also be obtained even in a case where the amount of thermal energy taken off by the blood flow differs or changes according to an individual difference, a difference in cured portion, and a difference in beat timing.

### Second Embodiment

Next, an ultrasonic energy therapy device and an ultrasonic energy therapy method according to a second embodiment of the present invention will be described below.

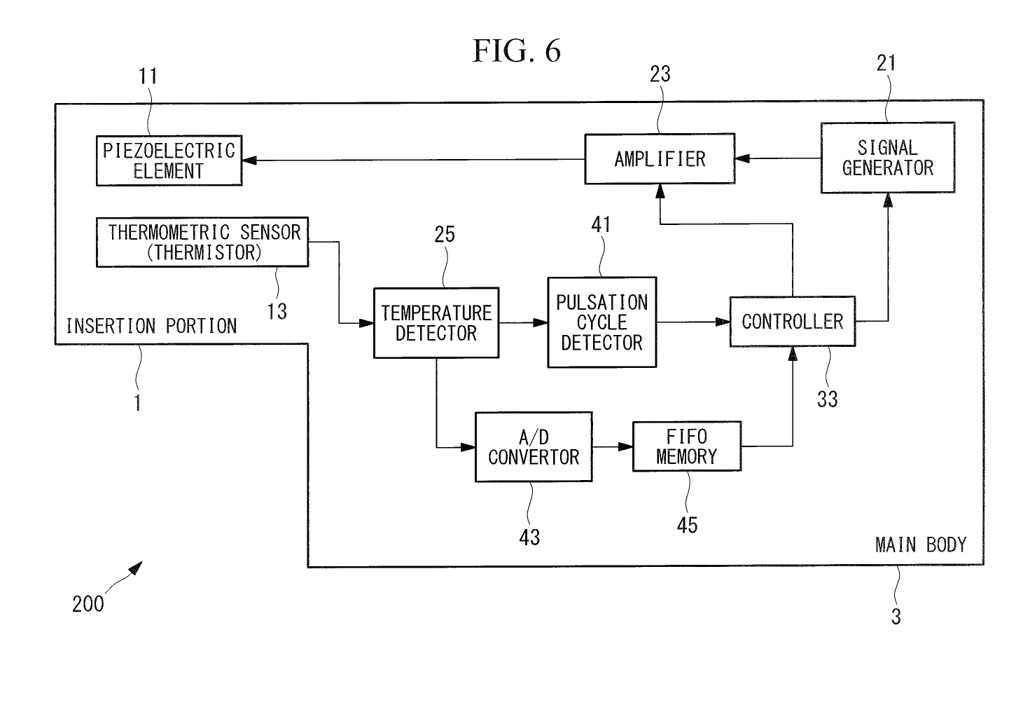

An ultrasonic energy therapy device 200 according to the present embodiment is different from that in the first embodiment in that the ultrasonic energy therapy device 200 comprises a pulsation cycle detector (pulsation detector) 41, an A/D convertor 43 and a FIFO (First In First Out) memory 45 in place of the smoothing circuit section 27, the comparison section 31, and the storage section 29, as illustrated in FIG. 6. The ultrasonic energy therapy method according to the present embodiment is different from that in the first embodiment in that the ultrasonic energy therapy method comprises a pulsation cycle detection step.

In the present embodiment, description is omitted with the same reference numerals to the components similar to those in the ultrasonic energy therapy device and the ultrasonic energy therapy method according to the first embodiment.

The temperature detection section 25 can send the temperature detection signal relating to the detection temperature of the thermometric sensor 13 to both of the pulsation cycle detection section 41 and the A/D conversion section 43.

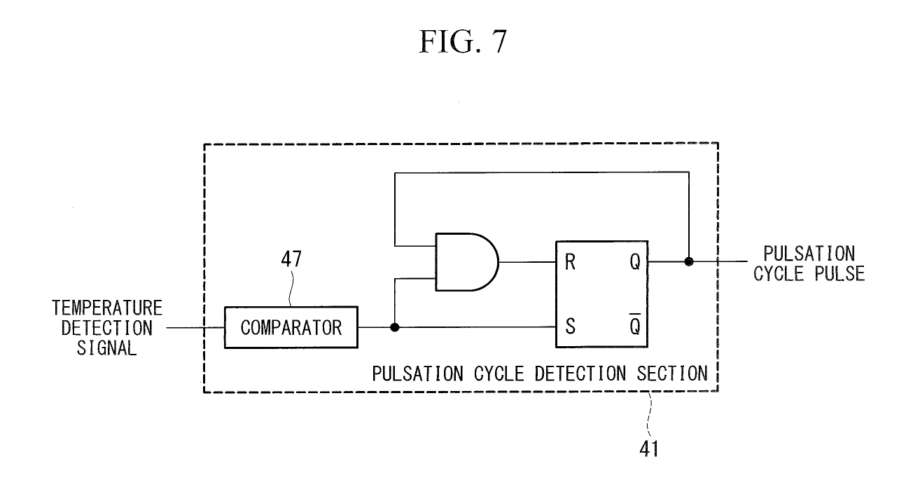

The pulsation cycle detection section 41 can detect a cycle of the pulsation based on the detection temperature of the thermometric sensor 13 sent from the temperature detection section 25. That is, the pulsation cycle detection section 41 comprises a comparator 47 as illustrated in FIG. 7, and the comparator 47 can compare the temperature detection signal of the thermometric sensor 13 sent from the temperature detection section 25 to generate a pulsation cycle pulse indicating the cycle of the pulsation. The pulsation cycle pulse generated by the pulsation cycle detection section 41 can be sent to the control section 33.

The A/D conversion section 43 can AD-convert the temperature detection signal of the thermometric sensor 13 sent from the temperature detection section 25.

The FIFO memory 45 temporarily can store the temperature detection signal AD-converted by the A/D conversion section 43 for one pulsation cycle in time sequence, and repeatedly update the signal every one pulsation cycle. The FIFO memory 45 can always store the temperature detection signal of one pulsation cycle.

The control section 33 can read the temperature detection signal of one pulsation cycle stored in the FIFO memory 45 in time sequence from the oldest. The control section 33 can generate an output control signal for emitting the ultrasonic energy having the intensity inversely proportional to the level of the temperature detection signal by synchronizing with a waveform of the pulsation cycle pulse sent from the pulsation cycle detection section 41 based on the temperature detection signal read from the FIFO memory 45.

Particularly, the control section 33, by synchronizing with the waveform of the pulsation cycle pulse, sends the output control signal for lowering the intensity of the ultrasonic energy to the amplification section 23 when the detection temperature of the thermometric sensor 13 rises, that is, the loss amount of the ultrasonic energy is decreased, and sends the output control signal for increasing the intensity of the ultrasonic energy to the amplification section 23 when the detection temperature of thermometric sensor 13 falls, that is, the loss amount of the ultrasonic energy is increased, so that the ultrasonic energy in a desired amount is radiated to the biological tissue.

The amplification section 23 can be adapted to change an amplification rate of a voltage applied to the piezoelectric element 11 based on the output control signal sent from the control section 33. Thus, the ultrasonic energy having the intensity inversely proportional to the level of the temperature detection signal before one pulsation cycle can be emitted from the piezoelectric element 11, by synchronizing with the waveform of the pulsation cycle pulse.

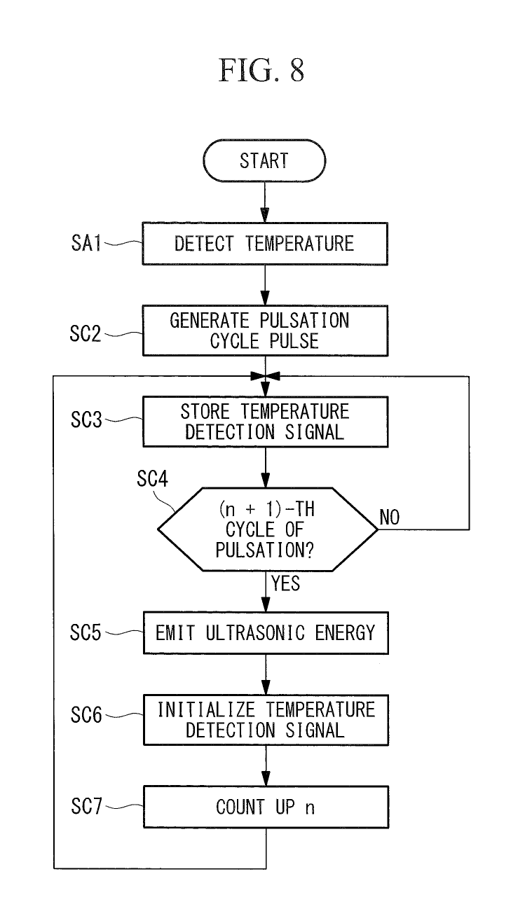

The ultrasonic energy therapy method according to the present embodiment comprises: a temperature detection step (step SA1, loss value detection step) that detects a change in a loss value with time of the ultrasonic energy emitted in the energy emission step (step SC5) caused by the blood flow, that is, the temperature of the thermometric sensor 13; and a pulsation cycle detection step (step SC2) that detects a pulsation cycle of the blood flow, as illustrated in FIG. 8.

The energy emission step, by synchronizing with the waveform of the pulsation cycle detected in the pulsation cycle detection step, lowers the intensity of the ultrasonic energy when the detection temperature of the thermometric sensor 13 detected in the temperature detection step rises, and increases the intensity of the ultrasonic energy when the detection temperature of the thermometric sensor 13 falls.

An operation of the ultrasonic energy therapy device 200 and the ultrasonic energy therapy method constituted in such a manner will be described with reference to a flow chart in FIG. 8.

To treat a lesion part of a patient by using the ultrasonic energy therapy device 200 and the ultrasonic energy therapy method according to the present embodiment, the thermometric sensor 13 is energized, and the insertion portion 1 is inserted into the blood vessel of the patient and is fixed in a positioning state by the balloons 19.

The temperature of the thermometric sensor 13 is detected by the temperature detection section 25 (step SA1, temperature detection step), the temperature detection signal is sent to the pulsation cycle detection section 41 and the A/D conversion section 43. In the pulsation cycle detection section 41, the temperature detection signal is compared by the comparator 47, and a pulsation cycle pulse is generated and sent to the control section 33 (step SC2, pulsation cycle detection step).

The temperature detection signal is AD-converted by the A/D conversion section 43 and the temperature detection signal of the n-th one pulsation cycle is stored in FIFO memory 45 in time sequence (step SC3).

Next, in the (n+1)-th cycle of pulsation (“Yes” in step SC4), the control section 33 reads the temperature detection signal of one pulsation cycle stored in the FIFO memory 45 in time sequence from the oldest.

The control section 33 sends the output control signal for emitting the ultrasonic energy having the intensity inversely proportional to the level of the temperature detection signal at the n-th pulsation to the amplification section 23 by synchronizing with a waveform of the (n+1)-th pulsation cycle pulse sent from the pulsation cycle detection section 41 based on the temperature detection signal of the n-th one cycle read from FIFO memory 45.

Particularly, the control section 33, by synchronizing with the waveform of the (n+1)-th pulsation cycle pulse, sends the output control signal for lowering the intensity of the ultrasonic energy to the amplification section 23 when the detection temperature of the thermometric sensor 13 rises, and sends the output control signal for increasing the intensity of the ultrasonic energy to the amplification section 23 when the detection temperature of thermometric sensor 13 falls, so that the ultrasonic energy in a desired amount is radiated to the biological tissue.

In the amplification section 23, an amplification rate of a voltage applied to the piezoelectric element 11 is changed based on the output control signal sent from the control section 33. Thus, the ultrasonic energy having the weak intensity is emitted from the piezoelectric element 11 when the temperature detection signal of the thermometric sensor 13 ascends, and the ultrasonic energy having the strong intensity is emitted from the piezoelectric element 11 when the temperature detection signal of the thermometric sensor 13 descends, by synchronizing with the waveform of the (n+1)-th pulsation cycle pulse (step SC5, energy emission step). That is, an output of the ultrasonic energy emitted by synchronizing with the waveform of the (n+1)-th pulsation cycle pulse corresponds to 1/(temperature detection signal at the n-th pulsation).

When the radiation of the (n+1)-th ultrasonic energy is finished, the temperature detection signal of the n-th one pulsation cycle stored in the FIFO memory 45 is initialized (step SC6). Then, n is counted up (step SC7) and the processing is returned to step SC3.

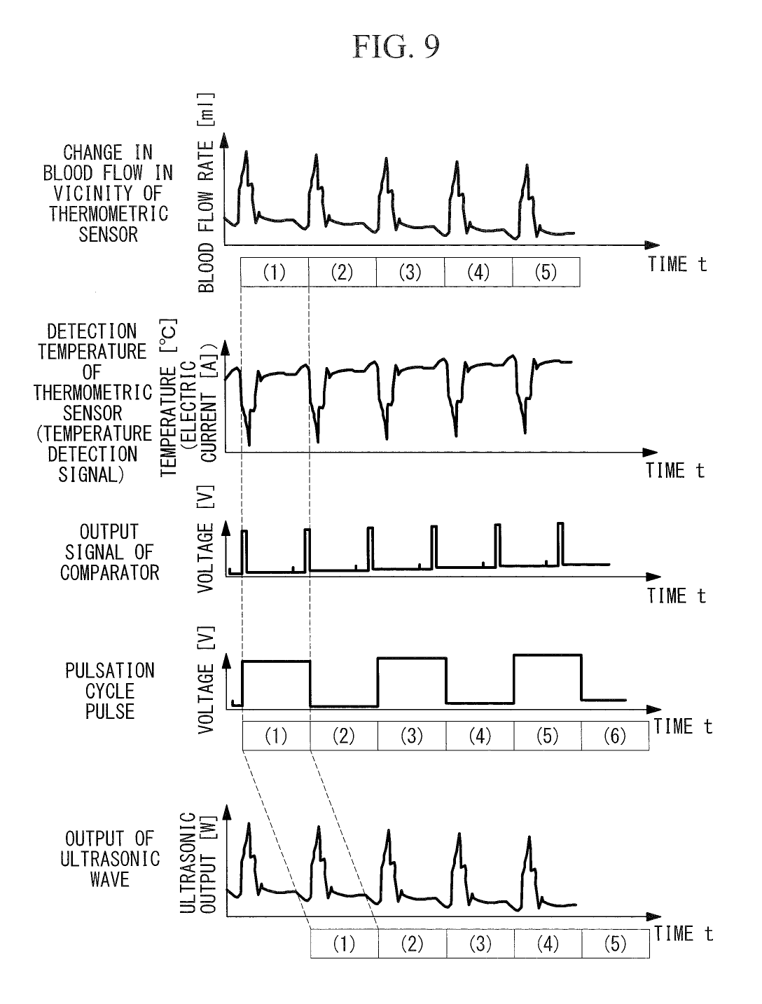

The blood flow volume and velocity largely change according to the pulsation. The blood flow is the fastest during systolic pulsation, and the blood flow is almost zero during diastolic pulsation. The temperature detection signal of the thermometric sensor 13 detected by the temperature detection section 25 (the loss amount of ultrasonic energy) also periodically changes as the pulsation periodically changes, as illustrated in FIG. 9. FIG. 9 illustrates a change in blood flow in the vicinity of a thermometric sensor 13, a detection temperature of the thermometric sensor 13, an output signal of the comparator 47, a pulsation cycle pulse and an output of the ultrasonic energy.

In the ultrasonic energy therapy device 200 and the ultrasonic energy therapy method according to the present embodiment, as illustrated in FIG. 9, the intensity of ultrasonic energy emitted from the piezoelectric element 11 is changed in inverse proportion to the level of the temperature detection signal before one pulsation cycle so that the ultrasonic energy in a desired amount is radiated to the biological tissue by synchronizing with the waveform of the pulsation cycle pulse, thereby preventing excessive radiation or insufficient radiation of the ultrasonic energy.

### Third Embodiment

Next, an ultrasonic energy therapy device and an ultrasonic energy therapy method according to a third embodiment of the present invention will be described below.

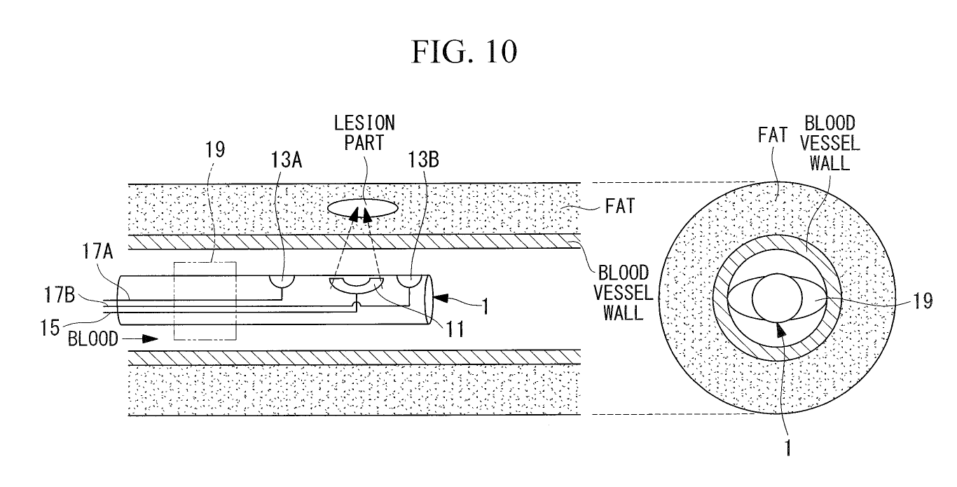

An ultrasonic energy therapy device 300 according to the present embodiment is different from that in the first embodiment in that two thermometric sensors 13A, 13B are provided in an insertion portion 1, as illustrated in FIG. 10.

In the present embodiment, description is omitted with the same reference numerals to the components similar to those in the ultrasonic energy therapy device and the ultrasonic energy therapy method according to the first embodiment.

The two thermometric sensors 13A, 13B are disposed at an interval from each other in a longitudinal direction of the insertion portion 1. The thermometric sensor 13A is disposed on a proximal end side of the insertion portion 1 than the piezoelectric element 11. The thermometric sensor 13B is disposed on a distal end side of the insertion portion 1 than the piezoelectric element 11. The piezoelectric element 11 is disposed substantially in a middle of these thermometric sensors 13A, 13B. The thermometric sensors 13A, 13B are connected to a main body 3 through signal lines 17A, 17B.

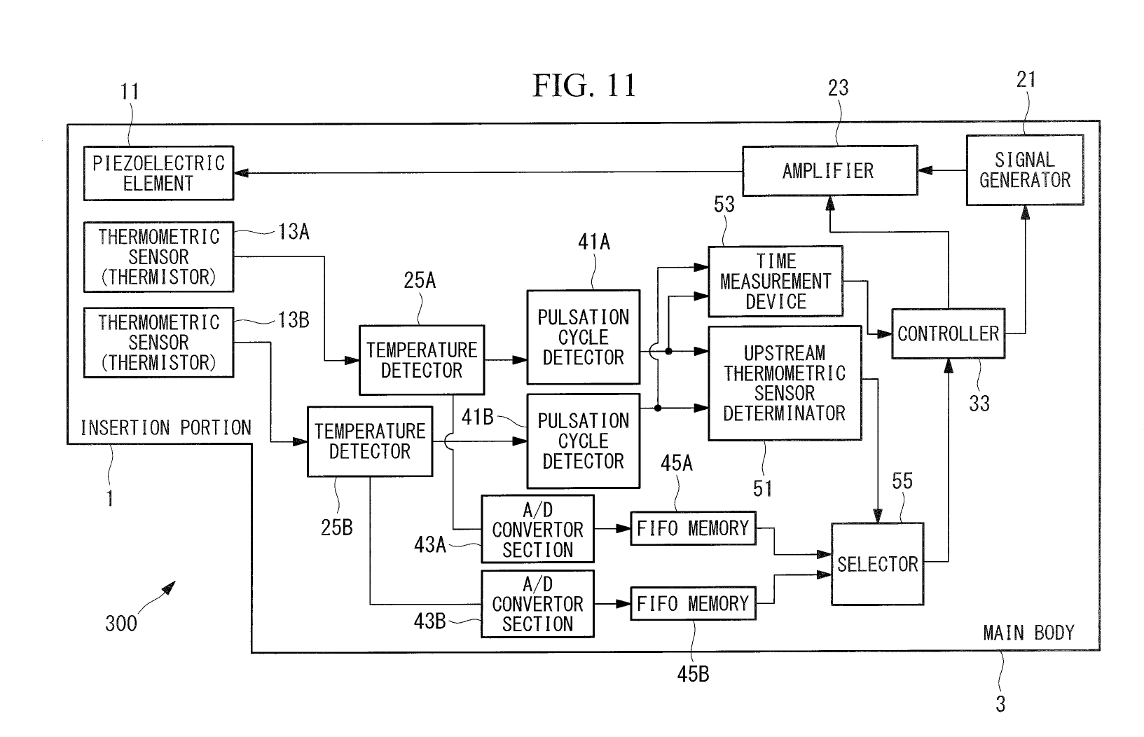

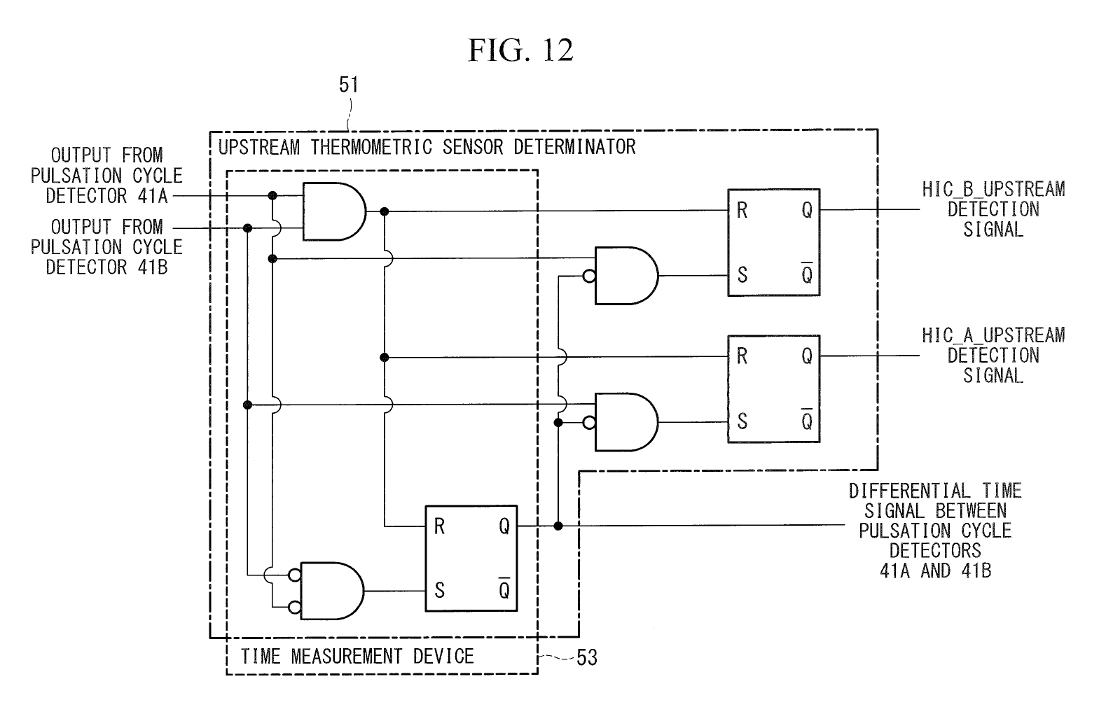

As illustrated in FIG. 11 and FIG. 12, the main body 3 comprises: a temperature detector 25A that can detect a temperature of the thermometric sensor 13A; a temperature detector 25B that can detect a temperature of the thermometric sensor 13B; a pulsation cycle detector 41A that can sample a temperature detection signal from the temperature detection section 25A; a pulsation cycle detector 41B that can sample a temperature detection signal from the temperature detection section 25B; an upstream thermometric sensor determinator 51 that can determine which of the thermometric sensors 13A, 13B is disposed on an upstream side of blood flow based on phases and timings of pulsation cycle pulses output from these pulsation cycle detection sections 41A, 41B; and a time measurement device 53 that can measure a time lag between temperature changes of the thermometric sensors 13A, 13B based on the phases and the timings of the pulsation cycle pulses output from these pulsation cycle detection sections 41A, 41B.

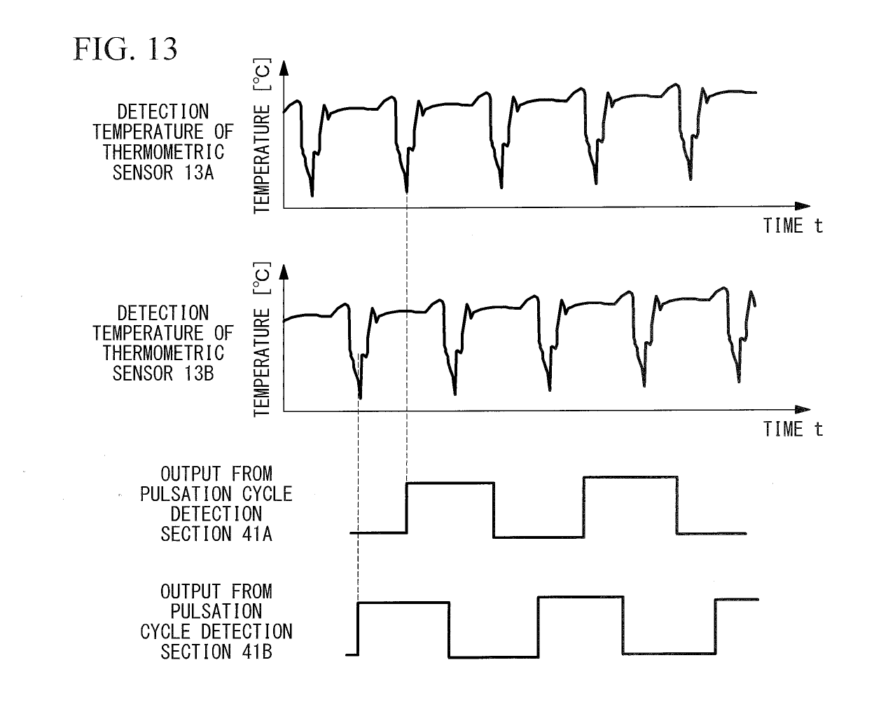

The pulsation cycle detection sections 41A, 41B can generate the pulsation cycle pulses indicating a cycle of the pulsation based on the sampled temperature detection signals from the temperature detection sections 25A, 25B, respectively. The blood flow largely changes according to the beat, and the temperatures of the thermometric sensors 13A, 13B also change. Since these thermometric sensors 13A, 13B are disposed apart from one another, a time lag occurs between the temperature changes detected by the thermometric sensors 13A, 13B as illustrated in FIG. 13. This time lag can be measured based on the phases and the timings of the pulsation cycle pulses of the pulsation cycle detection sections 41A, 41B.

The main body 3 comprises: an A/D convertor 43A that can AD-convert the temperature detection signal output from the temperature detection section 25A; an A/D convertor 43B that can AD-convert the temperature detection signal output from the temperature detection section 25B; a FIFO memory 45A that can temporarily store the temperature detection signal AD-converted by the A/D conversion section 43A for one pulsation cycle in time sequence; a FIFO memory 45B that can temporarily store the temperature detection signal AD-converted by the A/D conversion section 43B for one pulsation cycle in time sequence; and a selector 55 that can selectively read the temperature detection signal of the thermometric sensor 13A or 13B determined to be disposed on the upstream side by the upstream thermometric sensor determination section 51 from the FIFO memories 45A, 45B to send to the control section 33.

The control section 33 can generate an output control signal for emitting the ultrasonic energy having the intensity inversely proportional to the level of the temperature detection signal of the thermometric sensor 13A or 13B sent from the selector 55. Particularly, the control section 33 sends the output control signal for lowering the intensity of the ultrasonic energy to the amplification section 23 when the detection temperature of the thermometric sensor 13A or the thermometric sensor 13B rises, that is, the loss amount of the ultrasonic energy is decreased, and sends the output control signal for increasing the intensity of the ultrasonic energy to the amplification section 23 when the detection temperature of thermometric sensor 13 falls, that is, the loss amount of the ultrasonic energy is increased, so that the ultrasonic energy in a desired amount is radiated to the biological tissue.

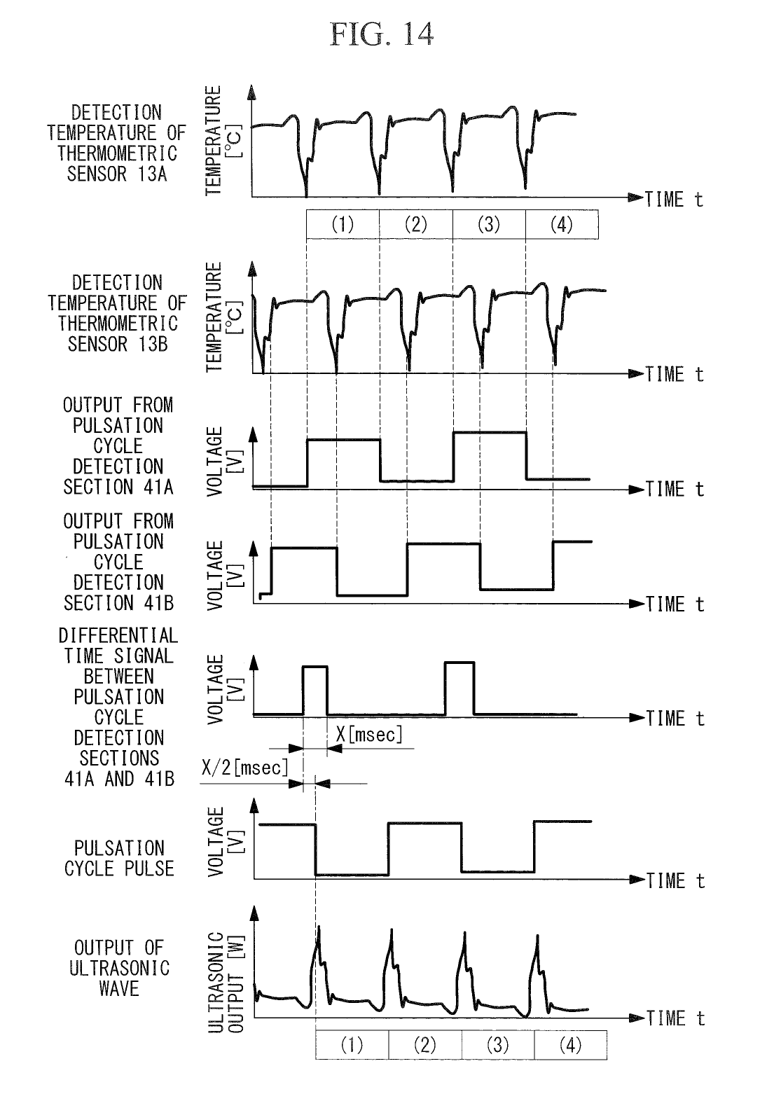

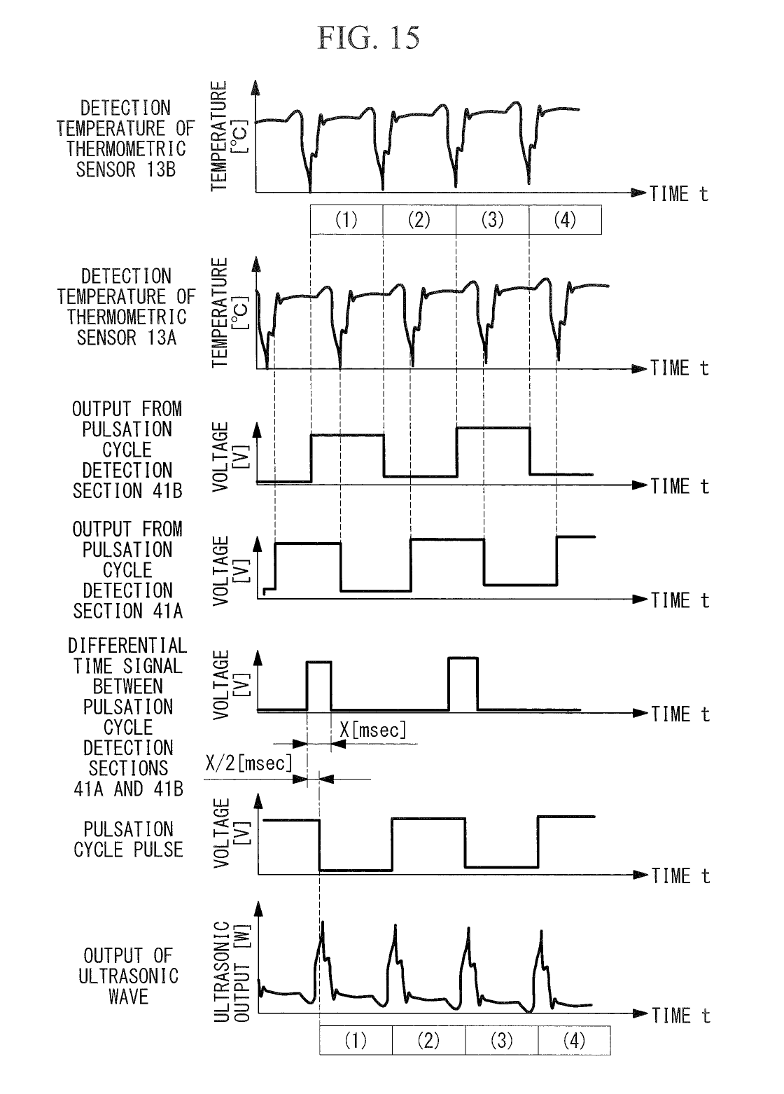

The control section 33 can adjust the timing of changing an amplification rate of a voltage by the amplification section 23 based on time lag information sent from the time measurement section 53. For example, when the time lag of the temperature changes of the thermometric sensors 13A, 13B is X (msec), the control section 33, based on the detection temperature of the thermometric sensor 13A or 13B which is disposed on the upstream side of the blood flow, delays the timing by X/2 (msec) after the pulsation cycle pulse of the pulsation cycle detection section 41A or the pulsation cycle detection section 41B changes and the amplification section 23 changes the amplification rate, as illustrated in FIG. 14 and FIG. 15.

Thus, as illustrated in FIG. 14 and FIG. 15, the intensity of the ultrasonic energy emitted from the piezoelectric element 11 can be changed by shifting by a time delay until a flow rate detection position in the blood where the flow rate is detected reaches the radiation position of the ultrasonic energy emitted from the piezoelectric element 11. FIG. 14 and FIG. 15 indicate the relations with a detection temperature of the thermometric sensor 13A, a detection temperature of the thermometric sensor 13B, an output of the pulsation cycle detection section 41A, an output of the pulsation cycle detection section 41B, a differential time signal between the pulsation cycle detection sections 41A and 41B, a pulsation cycle pulse, and an output of the ultrasonic energy, respectively. FIG. 14 illustrates an example of timing charts where the thermometric sensor 13A is disposed on the upstream side in the blood flow direction. FIG. 15 illustrates an example of timing charts where the thermometric sensor 13A is disposed on the upstream side in the blood flow direction.

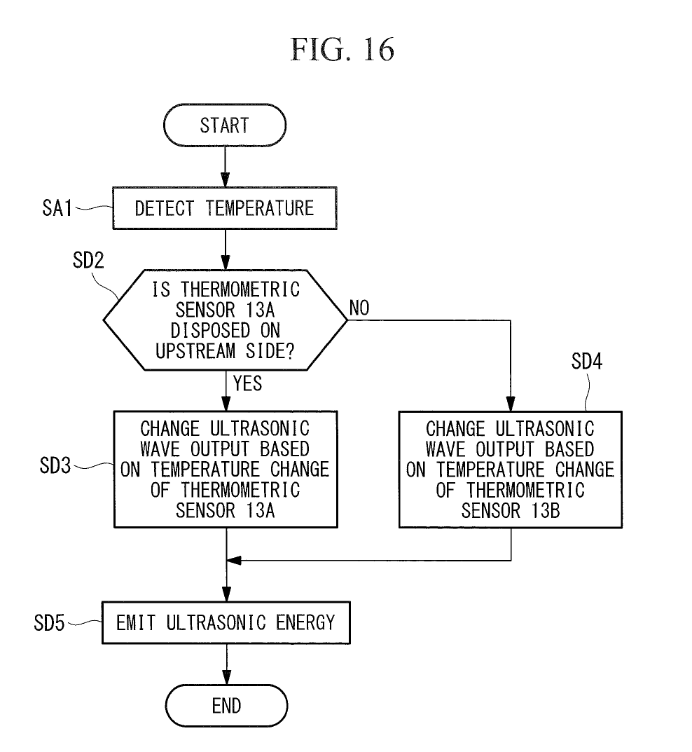

In the ultrasonic energy therapy method according to the present embodiment, as illustrated in FIG. 16, the temperature detection step (step SA1, loss value detection step) detects a change in a loss value with time of the ultrasonic energy, that is, the temperature of the thermometric sensor 13A or 13B which is disposed on the upstream side in the blood flow direction, based on a flow rate of blood detected on the upstream side in a blood flow direction from a radiation position of the ultrasonic energy emitted in the energy emission step.

The energy emission step (step SD5) adjusts the emission of the ultrasonic energy by shifting the timing by a time delay until the flow rate detection position in the blood where the temperature is detected in the temperature detection step reaches the radiation position of the ultrasonic energy emitted in the energy emission step, that is, by approximately half of the time lag between temperature changes of the thermometric sensors 13A, 13B measured by the time measurement section 53.

An operation of the ultrasonic energy therapy device 300 and the ultrasonic energy therapy method constituted in such a manner will be described with reference to a flow chart in FIG. 16.

To treat a lesion part of a patient by using the ultrasonic energy therapy device 300 and the ultrasonic energy therapy method according to the present embodiment, the thermometric sensors 13A, 13B are energized, and the insertion portion 1 is inserted into the blood vessel of the patient and is fixed in a positioning state by the balloons 19.

The temperatures of the thermometric sensors 13A, 13B are detected by the temperature detection sections 25A, 25B (step SA1), and each temperature detection signal is sent to the A/D conversion sections 43A, 43B and the pulsation cycle detection sections 41A, 41B. The temperature detection signals of the temperature detection sections 25A, 25B are AD-converted by the A/D conversion sections 43A, 43B, respectively, and stored for one pulsation cycle in time sequence in the FIFO memories 45A, 45B.

The pulsation cycle detection sections 41A, 41B sample the temperature detection signals from the temperature detection sections 25A, 25B, respectively, to generate the pulsation cycle pulses, and the pulsation cycle pulses are sent to the upstream thermometric sensor determination section 51 and the time measurement section 53.

The upstream thermometric sensor determination section 51 compares phases and timings of the pulsation cycle pulses sent from the pulsation cycle detection sections 41A, 41B (step SD2). As illustrated in FIG. 10, when the thermometric sensor 13A is disposed on the upstream side of the blood flow from the thermometric sensor 13B (“Yes” in step SD2), the control section 33 controls the amplification section 23 based on the temperature change of the thermometric sensor 13A (step SD3).

Particularly, the upstream thermometric sensor determination section 51 sends a determination result showing that the thermometric sensor 13A is disposed on the upstream side of the blood flow to the selector 55, and the selector 55 reads the temperature detection signal of one pulsation cycle of the thermometric sensor 13A stored in the FIFO memory 45A and sends it to the control section 33 in time sequence from the oldest.

The time measurement section 53 measures a time lag between temperature changes of the thermometric sensors 13A, 13B based on the phases and the timings of the pulsation cycle pulses output from these pulsation cycle detection sections 41A, 41B, and sends the obtained time lag information to the control section 33.

The control section 33, based on the temperature detection signal of the thermometric sensor 13A sent from the selector 55, sends the output control signal for emitting the ultrasonic wave output having the intensity inversely proportional to the level of the temperature detection signal to the amplification section 23, so that the ultrasonic energy in a desired amount is radiated to the biological tissue.

The control section 33 delays the timing when the amplification section 23 changes the amplification rate of the voltage by X/2 (msec) after the pulsation cycle pulse of the pulsation cycle detection section 41A changes, based on the time lag information sent from the time measurement section 53, as illustrated in FIG. 14.

With a delay by X/2 (msec) after the pulsation cycle pulse of the pulsation cycle detection section 41A changes, the ultrasonic energy having the weak intensity is emitted from the piezoelectric element 11 when the detection temperature of the thermometric sensor 13A rises, and the ultrasonic energy having the strong intensity is emitted when the detection temperature of the thermometric sensor 13A falls so that the ultrasonic energy in a desired amount is radiated to the biological tissue (step SD5).

On the other hand, when the thermometric sensor 13B is disposed on the upstream side of the blood flow (“No” in step SD2), the control section 33 controls the amplification section 23 based on the temperature change of the thermometric sensor 13B (step SD4).

Particularly, the upstream thermometric sensor determination section 51 sends a determination result showing that the thermometric sensor 13B is disposed on the upstream side of the blood flow to the selector 55. The selector 55 reads the temperature detection signal of one pulsation cycle of the thermometric sensor 13B stored in the FIFO memory 45B and sends it to the control section 33 in time sequence from the oldest.

The time measurement section 53 measures a time lag between temperature changes of the thermometric sensors 13A, 13B based on the phases and the timings of the pulsation cycle pulses output from these pulsation cycle detection sections 41A, 41B, and sends the obtained time lag information to the control section 33.

The control section 33, based on the temperature detection signal of the thermometric sensor 13B sent from the selector 55, sends the output control signal for emitting the ultrasonic wave output having the intensity inversely proportional to the level of the temperature detection signal to the amplification section 23, so that the ultrasonic energy in a desired amount is radiated to the biological tissue.

The control section 33 delays the timing when the amplification section 23 changes the amplification rate of the voltage by X/2 (msec) after the pulsation cycle pulse of the pulsation cycle detection section 41B changes, based on the time lag information sent from the time measurement section 53, as illustrated in FIG. 15.

With a delay by X/2 (msec) after the pulsation cycle pulse of the pulsation cycle detection section 41B changes, the ultrasonic energy having the weak intensity is emitted from the piezoelectric element 11 when the detection temperature of the thermometric sensor 13B rises, and the ultrasonic energy having the strong intensity is emitted when the detection temperature of the thermometric sensor 13B falls so that the ultrasonic energy in a desired amount is radiated to the biological tissue (step SD5).

As described above, in the ultrasonic energy therapy device 300 and the ultrasonic energy therapy method according to the present embodiment, the blood flow volume and velocity can be changed according to the beat timing and a patient's condition. The amount of thermal energy taken off by the blood flow in the ultrasonic energy also can be changed as the blood flow changes. The piezoelectric element 11 can be controlled at a timing corresponding to an actual change in blood flow, thereby preventing excessive radiation or insufficient radiation of the ultrasonic energy.

In the second embodiment and the third embodiment described above, the control section 33 can control the intensity of the ultrasonic energy emitted from the piezoelectric element 11, and the energy emission step adjusts the intensity of the ultrasonic energy. Alternatively, the control section 33 may control the emission time of the ultrasonic energy generated from the piezoelectric element 11 so that the ultrasonic energy in a desired amount can be radiated to the biological tissue. Also, the energy emission step may adjust the emission time of the ultrasonic energy so that the ultrasonic energy in a desired amount can be radiated to the biological tissue.

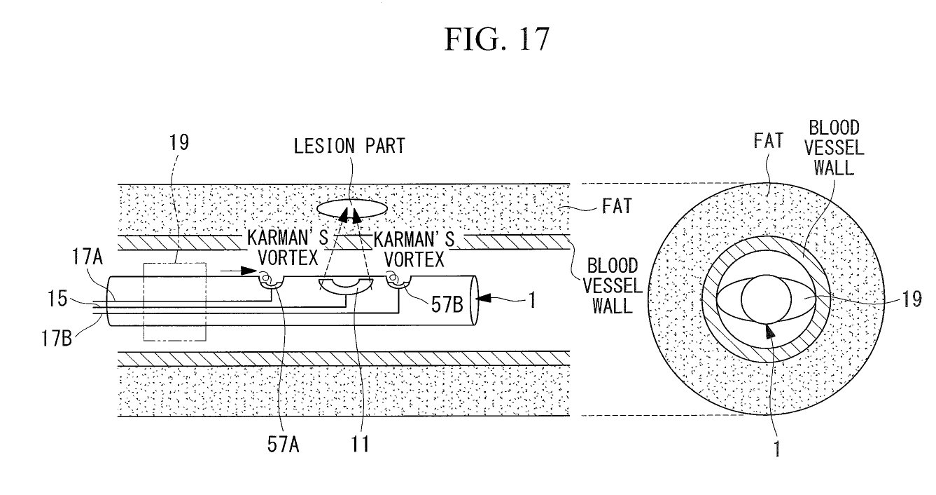

In the first embodiment, the second embodiment and the third embodiment, the thermometric sensors 13, 13A, 13B can be adopted as a means for detecting a flow rate of blood. Alternatively, an ultrasonic Doppler flow rate meter may be adopted to measure the flow rate of blood with ultrasonic waves, for example. In addition, as the means for detecting the flow rate of blood, a Karman's voltex type flow velocity sensors 57A, 57B may be adopted in place of the thermometric sensors 13, 13A, 13B, as illustrated in FIG. 17.

While the embodiments of the present invention have been described in detail with reference to the drawings, a specific configuration is not limited to these embodiments, and the present invention comprises design changes and the like without departing from the scope of the present invention. For example, the present invention is not limited to those described in the aforementioned each embodiment and modification, may be applied to embodiments made properly by combining these embodiments and modifications, and is not particularly limited.

As a result, the above-described embodiments lead to the following aspects.

A first aspect of the present invention is directed to an ultrasonic energy therapy device including: an insertion portion that has an elongated shape and is insertable into a blood vessel; an energy emission section that is attached to the insertion portion and emits ultrasonic energy from inside of the blood vessel toward a biological tissue outside the blood vessel; a loss amount measurement section that measures a loss amount of ultrasonic energy emitted from the energy emission section caused by blood flow; and a control section that controls the energy emission section according to the loss amount measured by the loss amount measurement section so that the ultrasonic energy in a desired amount is radiated to the biological tissue.

According to this aspect, a lesion part in the biological tissue outside the blood vessel is treated by inserting the insertion portion into the blood vessel and emitting the ultrasonic energy from the energy emission section. In this case, since the control section controls the energy emission section according to the loss amount of ultrasonic energy caused by the blood flow measured by the loss amount measurement section to radiate the ultrasonic energy in a desired amount to the biological tissue, the lesion part can be sufficiently treated regardless of a difference or a change in an amount of thermal energy taken off by the blood flow. Therefore, a certain therapeutic effect can be obtained even in a case where the amount of thermal energy taken off by the blood flow differs or changes according to an individual difference, a difference in cured portion, and a difference in beat timing.

In the above aspect, the ultrasonic energy therapy device comprises a comparison section that compares the loss amount measured by the loss amount measurement section and a predetermined first threshold. When the comparison section determines that the loss amount is larger than the predetermined first threshold, the control section may increase the intensity of the ultrasonic energy and/or prolong the emission time of the ultrasonic energy. When the comparison section determines that the loss amount is equal to or less than the predetermined first threshold, the control section may lower the intensity of the ultrasonic energy and/or shorten the emission time of the ultrasonic energy.

When the thermal energy taken off by the blood flow is large, an amount of ultrasonic energy radiated to the biological tissue becomes insufficient. On the other hand, when the thermal energy taken off by the blood flow is small, the amount of ultrasonic energy radiated to the biological tissue is not insufficient. Therefore, by setting a value which may discriminate between such situations as the predetermined first threshold, a lesion part can be treated by radiating the ultrasonic energy in the desired amount to the biological tissue based on a comparison result of the comparison section regardless of a difference in the amount of thermal energy taken off by the blood flow.

In the above aspect, when the comparison section determines that the loss amount is equal to or less than the predetermined first threshold, the comparison section compares the loss amount and a predetermined second threshold smaller than the predetermined first threshold. When the comparison section determines that the loss amount is equal to or less than the predetermined second threshold, the control section may stop the radiation of the ultrasonic energy.

When the thermal energy taken off by the blood flow is very small, that is, the blood flow does not affect substantially, a distance between the insertion portion and a blood vessel wall may not be maintained at a desired distance interval. Therefore, by setting a value which may recognize such a situation as the predetermined second threshold, the distance interval between the insertion portion and the blood vessel wall is shifted, thereby preventing the biological tissue not to be treated from being damaged by the radiation of the ultrasonic energy.

In the above aspect, the ultrasonic energy therapy device comprises a pulsation cycle detection section that detects a pulsation cycle of the blood flow. The control section, by synchronizing with a waveform of a pulsation cycle detected by the pulsation cycle detection section, may lower the intensity of the ultrasonic energy and/or shorten the emission time of the ultrasonic energy when the loss amount measured by the loss amount measurement section is reduced, and may increase the intensity of the ultrasonic energy and/or prolong the emission time of the ultrasonic energy when the loss amount is increased.

The blood flow volume and velocity largely change according to the pulsation. The blood flow is the fastest during systolic pulsation, and the blood flow is almost zero during diastolic pulsation. The loss amount of ultrasonic energy measured by the loss amount measurement section also periodically changes as the pulsation periodically changes. With such a constitution, the energy emission section is controlled following the change of the blood flow caused by the pulsation, thereby preventing excessive radiation or insufficient radiation of the ultrasonic energy.

In the above aspect, the loss amount measurement section may measure the loss amount based on a flow rate of blood detected on an upstream side in a blood flow direction from a radiation position of the ultrasonic energy emitted from the energy emission section. The control section may control the energy emission section by shifting the timing by a time delay until a flow rate detection position in the blood where the flow rate is detected by the loss amount measurement section reaches the radiation position of the ultrasonic energy emitted from the energy emission section.

The blood flow volume and velocity change according to the beat timing and a patient's condition. The amount of thermal energy taken off by the blood flow in the ultrasonic energy also changes as the blood flow changes. With such a constitution, the energy emission section is controlled at a timing corresponding to an actual change in blood flow, thereby preventing excessive radiation or insufficient radiation of the ultrasonic energy.

A second aspect of the present invention is directed to an ultrasonic energy therapy method including: an energy emission step of emitting ultrasonic energy from inside of a blood vessel toward a biological tissue outside the blood vessel; and a loss amount measurement step of measuring a loss amount of ultrasonic energy emitted in the energy emission step caused by blood flow. The energy emission step adjusts the emission of the ultrasonic energy according to the loss amount measured in the loss amount measurement step so that the ultrasonic energy in a desired amount is radiated to the biological tissue.

According to this aspect, a lesion part in the biological tissue outside the blood vessel is treated by emitting the ultrasonic energy from inside of the blood vessel in the energy emission step. In this case, since the energy emission step adjusts the emission of the ultrasonic energy according to the loss amount of ultrasonic energy caused by the blood flow measured by the loss amount measurement section to radiate the ultrasonic energy in a desired amount to the biological tissue, the lesion part can be sufficiently treated regardless of a difference or a change in an amount of thermal energy taken off by the blood flow. Therefore, a certain therapeutic effect can be obtained even in a case where the amount of thermal energy taken off by the blood flow differs or changes according to an individual difference, a difference in cured portion, and a difference in beat timing.

In the above aspect, the ultrasonic energy therapy method comprises a comparison step of comparing the loss amount measured in the loss amount measurement step and a predetermined first threshold. When the comparison step determines that the loss amount is larger than the predetermined first threshold, the energy emission step may increase the intensity of the ultrasonic energy and/or prolong the emission time of the ultrasonic energy. When the comparison step determines that the loss amount is equal to or less than the predetermined first threshold, the energy emission step may lower the intensity of the ultrasonic energy and/or shorten the emission time of the ultrasonic energy.

With such a constitution, by setting a value which may discriminate between excess and deficiency of the amount of ultrasonic energy radiated to the biological tissue as the predetermined first threshold, a lesion part can be treated by radiating the ultrasonic energy in the desired amount to the biological tissue based on a comparison result of the comparison step regardless of a difference in the amount of thermal energy taken off by the blood flow.

In the above aspect, when the comparison step determines that the loss amount is equal to or less than the predetermined first threshold, the comparison step compares the loss amount and a predetermined second threshold smaller than the predetermined first threshold. When the comparison step determines that the loss amount is equal to or less than the predetermined second threshold, the energy emission step may stop the radiation of the ultrasonic energy.

With such a constitution, by setting a value which may recognize a situation where a distance between an insertion portion and the biological tissue is not maintained at a desired distance interval as the predetermined second threshold, the distance interval between the insertion portion and the biological tissue is shifted, thereby preventing the biological tissue not to be treated from being damaged by the radiation of the ultrasonic energy.

A third aspect of the present invention is directed to an ultrasonic energy therapy method including: an energy emission step of emitting ultrasonic energy from inside of a blood vessel toward a biological tissue outside the blood vessel; and a loss value detection step of detecting a change in a loss value with time of ultrasonic energy emitted in the energy emission step caused by blood flow. When the loss value detected in the loss value detection step is reduced, the energy emission step lowers the intensity of the ultrasonic energy and/or shortens the emission time of the ultrasonic energy. When the detected loss value is increased, the energy emission step increases the intensity of the ultrasonic energy and/or prolongs the emission time of the ultrasonic energy.

According to this aspect, a lesion part can be treated by radiating the ultrasonic energy in the desired amount to the biological tissue according to the change of an amount of thermal energy taken off by the blood flow.

In the above aspect, the ultrasonic energy therapy method comprises a pulsation cycle detection step of detecting a pulsation cycle of the blood flow. The energy emission step controls the emission of the ultrasonic energy by synchronizing with a waveform of a pulsation cycle detected in the pulsation cycle detection step. When the loss value detected in the loss value detection step is reduced, the energy emission step may lower the intensity of the ultrasonic energy and/or shorten the emission time of the ultrasonic energy. When the detected loss value is increased, the energy emission step may increase the intensity of the ultrasonic energy and/or prolong the emission time of the ultrasonic energy.

With such a constitution, the amount of ultrasonic energy radiated to the biological tissue is controlled following the change of the blood flow, thereby preventing excessive radiation or insufficient radiation of the ultrasonic energy.

In the above aspect, the loss value detection step may detect the change in the loss value with time based on a flow rate of blood detected on an upstream side in a blood flow direction from a radiation position of the ultrasonic energy emitted in the energy emission step. The energy emission step may adjust the emission of the ultrasonic energy by shifting the timing by a time delay until a flow rate detection position in the blood where the flow rate is detected in the loss value detection step reaches the radiation position of the ultrasonic energy emitted in the energy emission step.

With such a constitution, the emission of the ultrasonic energy is controlled at a timing corresponding to an actual change in blood flow, thereby preventing excessive radiation or insufficient radiation of the ultrasonic energy.

According to the present invention, a certain therapeutic effect can be obtained even in a case where an amount of thermal energy taken off by blood flow differs or changes.

### REFERENCE SIGNS LIST

1 insertion portion
11 piezoelectric element (energy emission section)
13, 13A, 13B thermometric sensor (energy loss measurement section)
25, 25A, 25B temperature detection section (energy loss measurement section)
31 comparison section
33 control section
41 pulsation cycle detection section
100, 200, 300 ultrasonic energy therapy device
SA1 temperature detection step (loss amount measurement step, loss value detection step)
SA2, SA5 comparison step
SA4, SC5, SD5 energy radiation step
SC2 pulsation cycle detection step

1. An ultrasonic energy therapy device comprising:
an insertion portion that has an elongated shape and is insertable into a blood vessel;
an energy emitter that is attached to the insertion portion and emits ultrasonic energy from inside of the blood vessel toward a biological tissue outside the blood vessel;
a loss amount measurement device that measures a loss amount of ultrasonic energy emitted from the energy emission section caused by a blood flow; and
a controller that controls the energy emission section based on the loss amount measured by the loss amount measurement section so that the ultrasonic energy in a desired amount is radiated to the biological tissue.

2. The ultrasonic energy therapy device according to claim 1 comprising:
a comparator that compares the loss amount measured by the loss amount measurement section and a predetermined first threshold,
wherein when the comparison section determines that the loss amount is larger than the predetermined first threshold, the control section increases an intensity of the ultrasonic energy and/or prolongs an emission time of the ultrasonic energy, and
wherein when the comparison section determines that the loss amount is equal to or less than the predetermined first threshold, the control section lowers the intensity of the ultrasonic energy and/or shortens the emission time of the ultrasonic energy.

3. The ultrasonic energy therapy device according to claim 2,
wherein when the comparison section determines that the loss amount is equal to or less than the predetermined first threshold, the comparison section compares the loss amount and a predetermined second threshold smaller than the predetermined first threshold, and
wherein when the comparison section determines that the loss amount is equal to or less than the predetermined second threshold, the control section stops radiation of the ultrasonic energy.

4. The ultrasonic energy therapy device according to claim 1, further comprising:
a pulsation cycle detector that detects a pulsation cycle of the blood flow,
wherein the control section, by synchronizing with a waveform of a pulsation cycle detected by the pulsation cycle detection section, lowers the intensity of the ultrasonic energy and/or shortens the emission time of the ultrasonic energy when the loss amount measured by the loss amount measurement section is reduced, and increases the intensity of the ultrasonic energy and/or prolongs the emission time of the ultrasonic energy when the measured loss amount is increased.

5. The ultrasonic energy therapy device according to claim 1,
wherein the loss amount measurement section measures the loss amount based on a flow rate of blood detected on an upstream side in a blood flow direction from a radiation position of the ultrasonic energy emitted from the energy emission section, and
wherein the control section controls the energy emission section by shifting a timing by a time delay until a flow rate detection position in the blood where the flow rate is detected by the loss amount measurement section reaches the radiation position of the ultrasonic energy emitted from the energy emission section.

6. An ultrasonic energy therapy method comprising:
an energy emission step of emitting ultrasonic energy from inside of a blood vessel toward a biological tissue outside the blood vessel; and
a loss amount measurement step of measuring a loss amount of ultrasonic energy emitted in the energy emission step caused by blood flow,
wherein the energy emission step adjusts an emission of the ultrasonic energy according to the loss amount measured in the loss amount measurement step so that the ultrasonic energy in a desired amount is radiated to the biological tissue.

7. The ultrasonic energy therapy method according to claim 6, further comprising:
a comparison step of comparing the loss amount measured in the loss amount measurement step and a predetermined first threshold,
wherein when the comparison step determines that the loss amount is larger than the predetermined first threshold, the energy emission step increases an intensity of the ultrasonic energy and/or prolongs an emission time of the ultrasonic energy, and
wherein when the comparison step determines that the loss amount is equal to or less than the predetermined first threshold, the energy emission step lowers the intensity of the ultrasonic energy and/or shortens the emission time of the ultrasonic energy.

8. The ultrasonic energy therapy method according to claim 7,
wherein when the comparison step determines that the loss amount is equal to or less than the predetermined first threshold, the comparison step compares the loss amount and a predetermined second threshold smaller than the predetermined first threshold, and
wherein when the comparison step determines that the loss amount is equal to or less than the predetermined second threshold, the energy emission step stops radiation of the ultrasonic energy.

9. An ultrasonic energy therapy method comprising:
an energy emission step of emitting ultrasonic energy from inside of a blood vessel toward a biological tissue outside the blood vessel; and
a loss value detection step of detecting a change in a loss value with time of the ultrasonic energy emitted in the energy emission step caused by blood flow,
wherein when the loss value detected in the loss value detection step is reduced, the energy emission step lowers an intensity of the ultrasonic energy and/or shortens an emission time of the ultrasonic energy, and
wherein when the detected loss value is increased, the energy emission step increases the intensity of the ultrasonic energy and/or prolongs the emission time of the ultrasonic energy.

10. The ultrasonic energy therapy method according to claim 9, further comprising:
a pulsation cycle detection step of detecting a pulsation cycle of a blood flow,
wherein the energy emission step, by synchronizing with a waveform of a pulsation cycle detected in the pulsation cycle detection step, lowers the intensity of the ultrasonic energy and/or shortens the emission time of the ultrasonic energy when the loss value detected in the loss value detection step is reduced, and increases the intensity of the ultrasonic energy and/or prolongs the emission time of the ultrasonic energy when the detected loss value is increased.

11. The ultrasonic energy therapy method according to claim 9,
wherein the loss value detection step detects a change in the loss value with time based on a flow rate of blood detected on an upstream side in a blood flow direction from a radiation position of the ultrasonic energy emitted in the energy emission step, and
wherein the energy emission step adjusts an emission of the ultrasonic energy by shifting a timing by a time delay until a flow rate detection position in the blood where the flow rate is detected in the loss value detection step reaches the radiation position of the ultrasonic energy emitted in the energy emission step.
